(19) United States
(12) Patent Application Publication
(10) Pub. No.: US 2023/0009199 A1
(43) Pub. Date: Jan. 12, 2023

(54) STACKED FIXED BED BIOREACTOR AND METHODS OF USING THE SAME

(71) Applicant: CORNING INCORPORATED, CORNING, NY (US)

(72) Inventor: Vasiliy Nikolaevich Goral, Painted Post, NY (US)

(21) Appl. No.: 17/780,067

(22) PCT Filed: Nov. 4, 2020
(86) PCT No.: PCT/US2020/058764

§ 371 Date: May 26, 2022

Related U.S. Application Data
(60) Provisional application No. 62/941,308, filed on Nov. 27, 2019.

(51) Int. Cl. C12M 1/12; C12M 3/00; C12M 1/00

(52) U.S. Cl. CPC C12M 25/18; C12M 23/44; C12M 27/22; C12M 29/10

(57) ABSTRACT

A modular and stacked cell culture system that includes a standalone cell culture subunit with an interior cavity to house a cell culture substrate in a cell culture space, a fluid inlet to supply fluid to the cell culture space, and a fluid outlet to remove fluid from the cavity. The cavity is arranged for fluid to flow in from the fluid inlet, then through the cell culture space, and then out through the fluid outlet. The subunit further includes an alignment feature on at least one of a top and a bottom of the standalone cell culture subunit, wherein the alignment feature aligns with an alignment feature of another standalone cell culture subunit, such that multiple standalone cell culture subunits are stackable.

### CROSS REFERENCE TO RELATED APPLICATIONS

This application claims the benefit of priority under 35 U.S.C § 120 of U.S. Provisional Application Ser. No. 62/941,308 filed on Nov. 27, 2019, the content of which is relied upon and incorporated herein by reference in its entirety.

### FIELD OF THE DISCLOSURE

The present disclosure generally relates to the bioprocess field and, in particular, to a modular and stacked packed-bed bioreactor and a method for using the bioreactor for performing a cell culture.

### BACKGROUND

In the bioprocessing industry, large scale cultivation of cells is performed for purposes of the production of hormones, enzymes, antibodies, vaccines and cell therapies. A significant portion of the cells used in bioprocessing are anchorage dependent, meaning the cells need a surface to adhere to for growth and functioning. Traditionally, the culturing of adherent cells is performed on two-dimensional (2D) cell-adherent surfaces incorporated in one of a number of vessel formats, such as T-flasks, petri dishes, cell factories, cell stack vessels, roller bottles, and HYPERStack® vessels. These approaches can have significant drawbacks, including the difficulty in achieving cellular density high enough to make it feasible for large scale production of therapies or cells.

Alternative methods have been suggested to increase volumetric density of cultured cells. These include microcarrier cultures performed in stir tanks. In this approach, cells that are attached to the surface of microcarriers are subject to constant shear stress, resulting in a significant impact on proliferation and culture performance. Another example of a high-density cell culture system is a hollow fiber bioreactor, in which cells may form large three-dimensional aggregates as they proliferate in the interspatial fiber space. However, the cells growth and performance are significantly inhibited by the lack nutrients. To mitigate this problem, these bioreactors are made small and are not suitable for large scale manufacturing.

Another example of a high-density culture system for anchorage dependent cells is a packed bed bioreactor system. For example, packed bed bioreactor systems that contain a packed bed of support or matrix systems to entrap the cells have been previously disclosed U.S. Pat. Nos. 4,833,083; 5,501,971; and 5,510,262. Packed bed matrices usually are made of porous particles as substrates or non-woven microfibers of polymer. Such bioreactors function as recirculation flow-through bioreactors. One of the significant issues with such bioreactors is the non-uniformity of cell distribution inside the packed bed. For example, the packed bed functions as depth filter with cells predominantly trapped at the inlet regions, resulting in a gradient of cell distribution during the inoculation step. In addition, due to random fiber packaging, flow resistance and cell trapping efficiency of cross sections of the packed bed are not uniform. For example, medium flows fast though the regions with low cell packing density and flows slowly through the regions where resistance is higher due to higher number of entrapped cells. This creates a channeling effect where nutrients and oxygen are delivered more efficiently to regions with lower volumetric cells densities and regions with higher cell densities are being maintained in suboptimal culture conditions. Another significant drawback of packed bed systems disclosed in a prior art is the inability to efficiently harvest intact viable cells at the end of culture process. U.S. Pat. No. 9,273,278 discloses a bioreactor design to improve the efficiency of cell recovery from the packed bed during cells harvesting step. It is based on loosening the packed bed matrix and agitation or stirring of packed bed particles to allow porous matrices to collide and thus detach the cells. However, this approach is laborious and may cause significant cells damage, thus reducing overall cell viability.

All existing platforms based on packed-bed bioreactors have the limitation that, when the cell density increases towards its maximum level, the cells at the rear end of the bioreactor (with respect to the flow path through the bioreactor) cannot obtain enough nutrition or oxygen, and cell productivity will thus be inhibited. This depletion of nutrients or oxygen can be viewed as a gradient of nutrient and/or oxygen supply through the flow path of the packed-bed. To reduce the development of such a nutrient/oxygen gradient that is detrimental to cell functionality, fixed beds can be designed to have relatively short media perfusion path. However, such designs significantly impact the reactor scalability in bioprocess therapeutics manufacturing. For example, while suspension stirred tank bioreactors can be scaled up to 2,000 L or to 10,000 L, typical packed bed bioreactors are only scalable up to 50 L of capacity. While manufacturing of viral vectors for early-phase clinical trials is possible with existing platforms, there is a need for a platform that can produce high-quality product in greater numbers in order to reach late-stage commercial manufacturing scale. In particular, there is a need for a platform and methods for compartmentalizing the packed bed while managing fluid flow of cells and nutrients through the bed, and reducing nutrient and/or oxygen gradients through the packed bed.

### SUMMARY

Disclosed herein is a modular cell culture system having a standalone cell culture subunit with an interior cavity to house a cell culture substrate in a cell culture space, a fluid inlet to supply fluid to the cell culture space, a fluid outlet to remove fluid from the interior cavity, and at least one alignment feature disposed on at least one of a top and a bottom of the standalone cell culture subunit. The interior cavity is arranged to allow fluid to flow in from the fluid inlet, through the cell culture space, and out through the fluid outlet. The at least one alignment feature can align with at least one alignment feature of another standalone cell culture subunit, such that the standalone cell culture subunit is stackable with another standalone cell culture subunit. According to embodiments, the system includes a plurality of the standalone cell culture subunit, the plurality of the standalone cell culture subunits being stacked together.

Additional aspects of the present disclosure will be set forth, in part, in the detailed description, figures and any claims which follow, and in part will be derived from the detailed description, or can be learned by practice of the disclosure. It is to be understood that both the foregoing general description and the following detailed description are exemplary and explanatory only and are not restrictive of the disclosure as disclosed.

### BRIEF DESCRIPTION OF THE DRAWINGS

A more complete understanding of the present disclosure may be had by reference to the following detailed description when taken in conjunction with the accompanying drawings wherein:

FIG. 1 illustrates a schematic cross-section view of a cell culture subunit, according to one or more embodiments of this disclosure;

FIG. 2 illustrates a stack of the modular cell culture subunits of FIG. 1, according to one or more embodiments of this disclosure;

FIG. 3 illustrates a schematic cross-section view of a cell culture subunit, according another embodiment of this disclosure;

FIG. 4 illustrates a stack of the modular cell culture subunits of FIG. 3, according to one or more embodiments of this disclosure;

FIGS. 5A and 5B show a detailed view of a retaining feature inside the cell culture subunit, according to one or more embodiments of this disclosure;

FIG. 6 illustrates a cell culture system and flow path using a modular cell culture stack with separate media conditioning vessels, according to one or more embodiments of this disclosure;

FIG. 7 illustrates a cell culture system and flow path using a modular cell culture stack and common media conditioning vessel, according to one or more embodiments of this disclosure;

FIG. 8A shows a plan view of a radial flow manifold of a modular cell culture subunit, according to one or more embodiments of this disclosure;

FIG. 8B shows a cross-section schematic of the radial flow manifold of FIG. 8A with a cell culture substrate, according to one or more embodiments of this disclosure;

FIG. 9 illustrates a cross-section schematic of a stacked arrangement of cell culture subunits of FIGS. 8A and 8B, according to one or more embodiments of this disclosure;

FIG. 10 shows the stacked arrangement of FIG. 9 in a carrier with aseptic pull tabs, according to one or more embodiments of this disclosure;

FIG. 11 shows a stacked arrangement of two carriers of FIG. 10, according to one or more embodiments of this disclosure;

FIG. 12 shows a larger stacked arrangement of carriers of FIG. 10, according to one or more embodiments of this disclosure;

FIG. 13 is a schematic illustrating a bioprocessing system incorporating a packed-bed bioreactor according to embodiments of the present disclosure; and

FIG. 14 shows an operation for controlling a perfusion flow rate of a cell culture system, according to one or more embodiments.

### DETAILED DESCRIPTION

Various embodiments of the present disclosure will be discussed with reference to the figures, which illustrate various aspects of packed-bed bioreactor systems and related methods of using the bioreactor systems according to non-limiting embodiments of the present disclosure. The following description is intended to provide an enabling description of the bioreactor systems and the various aspects of the bioreactor systems and methods will be specifically discussed in detail throughout the disclosure with reference to the non-limiting embodiments, these embodiments are interchangeable with one another within the context of the disclosure.

The present disclosure describes a modular fixed-bed bioreactor and bioreactor system having a stack of such fixed-bed bioreactors. Embodiments include the individual modules of fixed-bed bioreactors, as well as a stacked assembly of connecting units. Using individual packed-bed modules that can be combined provides a solution that is scalable and removes the limitations of operational conditions imposed by nutrient and/or oxygen gradients within the packed bed during cell culture. Each individual module provides a short media perfusion path and thus supports optimal cell culture conditions. Multiple individual modules can be assembled into one unit thus providing scale up flexibility of the manufacturing process. Depending on the targeted yield of the production batch, an end user can configure a system to utilize 1 to 10 or more individual units simultaneously, for example.

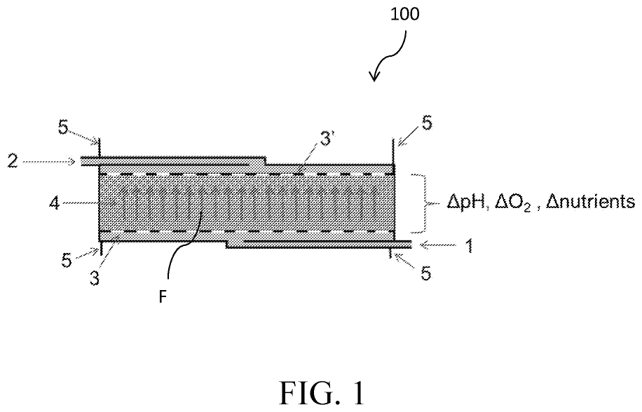

Referring to FIG. 1, an individual cell culture unit 100 of a module cell culture system is shown, according to one or more embodiments of this disclosure. The cell culture unit 100 includes a vessel having a cell culture media inlet 1 and a cell culture media outlet 2 to perfuse media during bioreactor operation. The system 100 also includes flow distribution plates 3, 3′ provided on either side of a packed bed layer of a cell culture substrate 4 to assist in uniform mead perfusion in the pack bed. The flow distribution plates 3, 3′ may also be structural reinforcement or containment barriers for the packed be to keep the cell culture substrate in place and structurally supported. With the arrangement of the cell culture substrate 4 between the inlet 1 and outlet 2, media flows through the substrate 4 in the direction F indicated by the arrows in the substrate 4. During the bioreactor operation, adherent cells attach and grow on a surface of the substrate 4 packed in the bioreactor bed. The unit 100 is designed so that the packed bed has a short height to minimize nutrient and oxygen gradient development along the media flow path F through the bed.

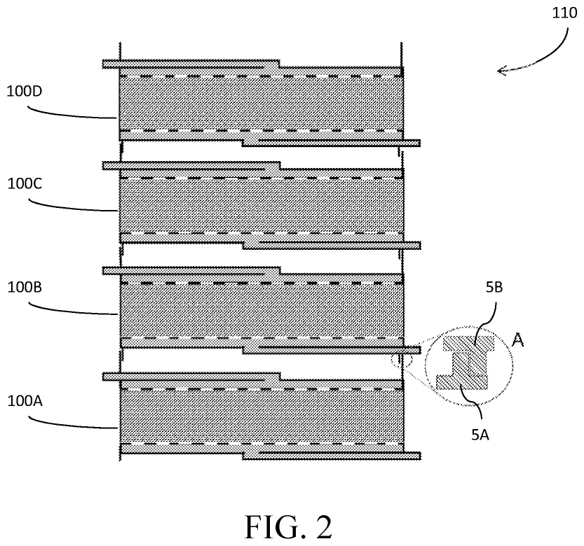

The cell culture unit 100 further includes one or more alignment features 5 provided on the top and/or bottom of the unit 100. The alignment features 5 are used to align multiple cell culture units, similar to cell culture unit 100, so that the bioreactor system can be scaled up. FIG. 2 shows an example of one embodiment in which four units 100A-100D are stacked in this manner to form a stacked cell culture system 110. Insert A in FIG. 2 shows how the alignment features 5A and 5B of adjacent units 100A and 100B, respectively. Thus, the alignment features ensure that the units can be securely stacked and aligned. As an aspect of some embodiments, the alignment features can be used to secure a top or cap to the unit 100 when it is used as a single cell culture unit or when it is the top-most unit of a stack. The alignment features can take a number of forms, including the simple straight walls shown in FIGS. 1 and 2, and can also include features that interlock to prevent unintended separation of the stack.

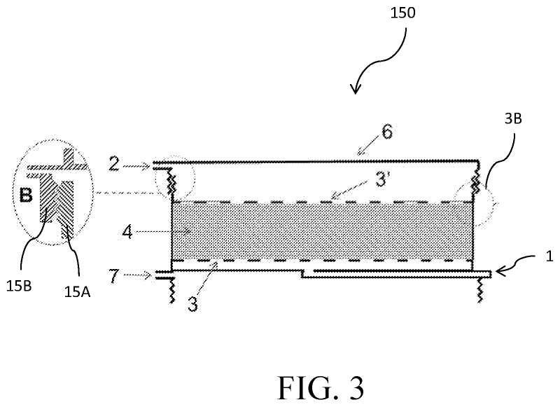

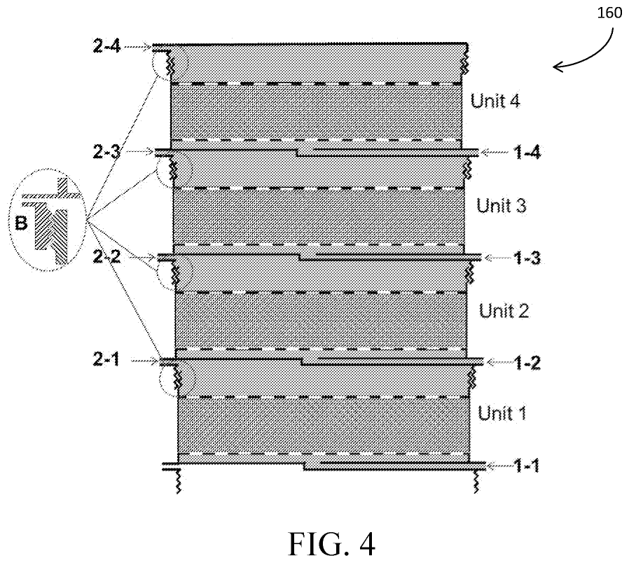

For example, FIG. 3 shows a cell culture unit 150 according to an embodiment in which alignment features 15A and 15B have interlocking profiles to prevent unintended separation, as shown in area 3B and insert B. In addition, as shown in the insert B of FIG. 3, a top lid 6 is provided that has an integrated outlet port 2. Similarly, the alignment features provided on the bottom of cell culture unit 150 have an integrated outlet port 7 designed to be an outlet port for a cell culture vessel stacked beneath unit 150. In this way, the individual units can be efficiently stacked, and each unit can have inlet and outlet ports without the need for excess material or bulk to provide a scalable but compact cell culture system. For example, stacking four of the units 150 will result in a cell culture system 160 as shown in FIG. 4. In the system 160, four individual units having the construction shown in FIG. 3 are stacked and integrated. Each of units 1 through 4 in FIG. 4 has a media inlet (1-1, 1-2, 1-3, and 1-4, respectively) and a media outlet (2-1, 2-2, 2-3, and 2-4, respectively). A detailed view of the media outlets is shown in insert B.

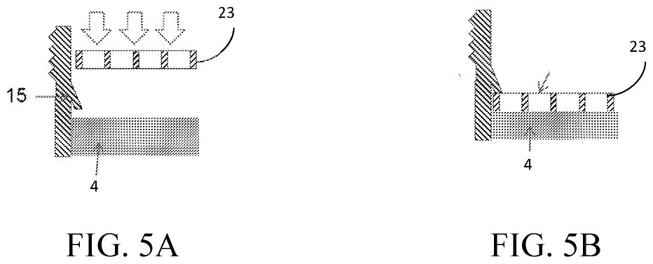

FIGS. 5A and 5B show an aspect of one or more embodiments of the cell culture units, where a flow distribution plate 23 or retention screen can be help in place above the cell culture substrate 4 by being placed under a retention feature 15. In addition to holding the packed bed substrate in place, the retention screen or flow distribution plate 23 can be used to compress the packed bed to a desired packing density. For example, in FIG. 5A, the packed bed is uncompressed because the retention screen has not been put in place, resulting in a relatively loose packing density. However, when the retention screen 23 is snapped in place under the retention feature 15, the bed can be compressed. The thickness of the retention feature 15 can be varied to achieve the desired packing density for a given application, or multiple retention screens can be used to increase packing density, according to some embodiments.

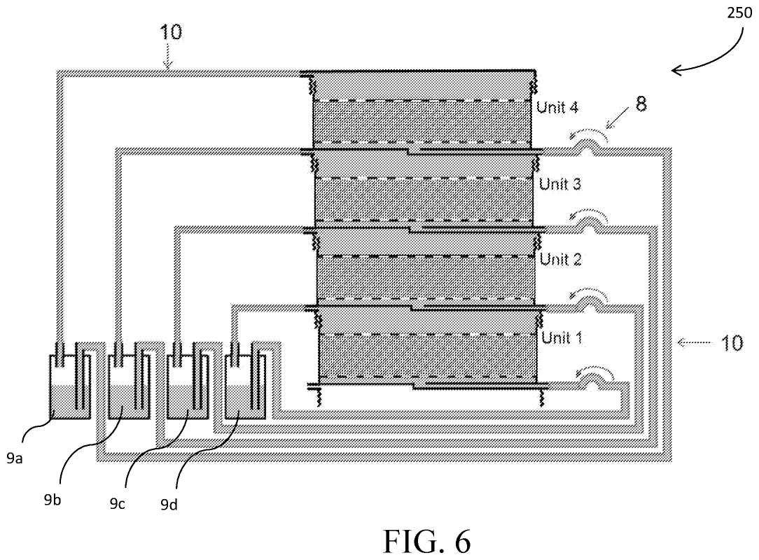

FIG. 6 shows a stacked cell culture system 250, according to some embodiments. The system 250, as shown, has four individual cell culture units 1 through 4 stacked in the manner shown in FIG. 4. Cell culture media in each unit is perfused through flexible tubing 10 independently by designated pumps 8. The cell culture media of each unit is conditioned by adjusting any of a number of factors, including pH, Temperature, dissolved oxygen, etc. In the embodiment of FIG. 6, this conditioning occurs independently in designated media conditioning vessels 9a-9d.

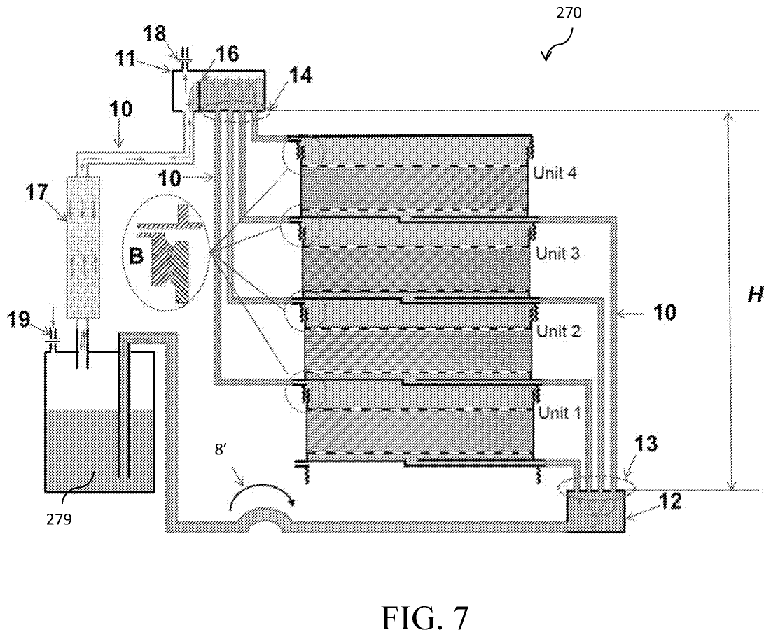

FIG. 7 shows another embodiment of this disclosure in which a stacked cell culture system 270. The individual units 1 through 4 of the system 270 are stacked similarly to those of FIGS. 4 and 6. However, in system 270, the units 1-4 are operated in parallel using a common media conditioning vessel 279. Cell culture media in each unit 1-4 is perfused through flexible tubing 10 by designated pump 8′. Media flow is equally distributed between individual units in flow splitter 12. Equal flow resistance between bioreactor units is achieved by hydrostatic pressure equilibration in vessel 11. After passing vessel 11, media flows by gravity back to media conditioning vessel 279 through the oxygenation column 17 where oxygen is bubbled and dissolved into the media. Optionally, the system 270 is open to the atmosphere at the gas outlet 18.

According to the above-described aspects of embodiments of this disclosure, a packed-bed bioreactor system for anchorage dependent cells is provided that can scale up easily to any practical production scale for cells or cell-derived products (e.g., proteins, antibodies, viral particles). In one embodiment, individual bioreactor subunits are packed with structurally defined cell culture substrate on which adherent cells can attach, proliferate and function. In a preferred embodiment, the cell culture substrate is a woven polymer material or mesh, or a stack of woven sheets.

As discussed above, different aspects of embodiments of these cell culture subunits are presented in FIGS. 1 and 3. For example, each bioreactor subunit has media inlet port 1, media outlet port 2, and packed bed region 4 for adherent cell attachment, proliferation and functioning. To ensure stable and uniform delivery of nutrients and oxygen to cells adherent in the packed bed bioreactor, uniform and/or constant flow of media is required through the bioreactor subunit. Media perfusion uniformity is achieved by incorporating flow redistribution plate 3 in beneath of upstream of the packed bed (FIGS. 1 and 3). The flow redistribution plate positioned at the media inlet redirects the media flow from central inlet point into horizontal direction so as to ensure uniform media perfusion through the packed bed (arrows F in FIG. 1). Because of the pack flow nature of media perfusion through the packed bed bioreactor, nutrients, pH and oxygen gradients are developed along the packed bed. This fact poses a limitation to the overall height of the packed bed. In general, typical packed bed heights for adherent cell cultures are a maximum of 10 cm due to the restraint imposed by the depleted media gradient. To increase the production capacity of packed bed bioreactors beyond what is possible with a single 10 cm packed bed, the concept of modular and stackable subunits is disclosed herein. The packed bed height of individual bioreactor subunit can be constant and at a thickness small enough (e.g., about 20 cm or less, or about 10 cm or less) that the depleted media gradient is a non-issue in the individual subunits.

To minimize a footprint of the system in a production facility, multiple subunits can be vertically integrated into single system. FIGS. 2 and 4 each show four individual subunits stacked on top of each other to form stacked systems 110 and 160. To ensure physical stability of the assembly, individual unit are aligned with interlocking rims (see e.g., insert A in FIG. 2) protruding above the top and bottom surfaces of the subunits. Alternatively, individual subunits can be interlocked by using threaded connections located on two identical subunits (see, e.g., insert B in FIGS. 3 and 4). If it is desired to operate one subunit as a stand-alone unit, its top can be replaced with the lid 6 having a built-in outlet 2, as shown in FIG. 3.

FIG. 4 shows four subunits vertically integrated by using threaded connections. Each subunit has individual media inlet 1 and outlet 2 ports. The bottom portion of each subunit serves as a top part of a lower subunit. Each subunit has a cell culture region with a packed bed woven substrate for adherent cell growth and production of therapeutic ingredients. As discussed above, the woven mesh can be packed, compressed, and retained in the bioreactor by the means of retaining grid 3′ as depicted in FIGS. 5A and 5B. After the mesh substrate 4 has been stacked in the bioreactor cavity, the retaining grid 3′ is pressed and locked in the position by retaining feature 15 protruding from the inner wall of the bioreactor.

The vertically stacked individual bioreactor subunits shown in FIGS. 2 and 4 can be operated independently by being perfused by individual pumps 8 and by having individual media conditioning vessels 9 (as shown in FIG. 6). Such a setup can be particularly applicable at the stage of bioprocess development. Simultaneously running identical bioreactors while having a flexibility of maintaining different cell culture conditions (pH, DO, CO₂ level, media composition, feeding schedule) will allow end users to quickly identify the optimal bioprocess conditions.

In production process, individual bioreactor subunits can be operated in parallel by utilizing single perfusion pump 8′ and common media conditioning vessel 279, as shown in FIG. 7. To have uniform and equal media perfusion in all bioreactor subunits, a unique fluid flow path is presented as an aspect of this embodiment. In particular, after the cell culture media is perfused through individual bioreactor subunits by liquid pump 8′, the media enters flow distribution unit 12, as shown in FIG. 7. Diameters of all media outlets from this unit to the bioreactor subunits are equal to provide equal flow resistance. FIG. 7 depicts the flow distribution unit 12 as having four media outlets for four individual bioreactor subunits (Unit 1-Unit 4), for example. However, embodiments include systems with fewer or more media outlets and bioreactor subunits. The volumetric flow rate through each individual bioreactor subunit for steady-state pressure-driven flow can be presented as

Q=ΔP/R

where ΔP is the pressure difference between media inlet 13 and outlet 14 in FIG. 7. This pressure difference consists of frictional pressure loss due to resistance of the fluid to movement and hydrostatic pressure loss due to gravity force and the elevation difference H between media inlet 13 and outlet 14. For all four bioreactor subunits, media inlets 13 and outlets 14 are positioned at the same levels, thus hydrostatic pressure loss will be the same. When all bioreactor subunits have the same volume of packed bed and the total length of the tubing 10 is the same, then hydrodynamic pressure will be the same. As result of this, the total pressure difference between media inlet 13 and media outlet 14 will be the same for all individual bioreactor subunits (Unit 1-Unit 4 in FIG. 7). This means that a single fluid pump 8′ can provide uniform media perfusion in all subunits during regular cell culture. Identical media perfusion rates in all bioreactor subunits will provide identical cell culture conditions and will require only one media conditioning vessel 279 to maintain proper pH, oxygenation, temperature and nutrients concentration in the media.

To equilibrate hydrostatic pressure at the outlet, overflow unit 11 may also be provided. It has tubing outlets positioned at the same level as inlets 14 and a media overflow barrier 16. Media overflow barrier allows media to be fed by gravity back into the media conditioning vessel 279. Overflow unit is opened to the outside atmosphere via the port 18 while sterility of the system is maintained by sterile gas filter. Allowing the media free low into media conditioning vessel significantly increases the area of gas/liquid interface. In addition, on the return path media passes through oxygenation column 17 to replenish depleted levels of dissolved oxygen. Premixed gas supply 19 can also be introduced into bioreactor conditioning vessel 279 to re-oxygenate media. This gas (indicated by blue arrows) creates counter flow in the oxygenation column and exits the system at outlet 18. Having the oxygenation gas in counterflow to the media in oxygenation column 17 can significantly increase oxygen concentration in depleted media.

As an additional aspect of some embodiments of this disclosure, packed bed bioreactor vessels and systems are provided that use radial flow of cell culture media through the packed bed. Such embodiments provide a fixed bed bioreactor with uniform flow velocity within the bioreactor's packed bed cell culture substrate to create a controlled and uniform environment throughout multiple fixed beds in the entire modular system. The system is further designed to reduce non-bed volume within the bioreactor such that the footprint of the bioreactor is as compact as possible. Still further, it is designed to be modular so that an end user can optimize the system size to their manufacturing needs. It is also designed to be a closed system.

According to one or more embodiments, thin radial flow manifolding is positioned between fixed beds subunits within which cells are cultured. The flow rate within the radial flow manifolds varies due to resistance in the bioreactor design such that media is delivered to the fixed bed at uniform rates across a surface and through the bulk of the fixed bed(s). Because units can become very heavy when loaded with substrate material and media, the modular design allows very large single use fixed bed bioreactors to be created manually by limiting the weight of the modules to safe handling limits for operators, e.g., less than 25 lbs for lifting purposes. To aid in making the vessel easy to use and to maintain the closed system nature of the vessel aseptic connections can be used which can either be built into the modules or can be supplied onto tubing on the perimeter of the module.

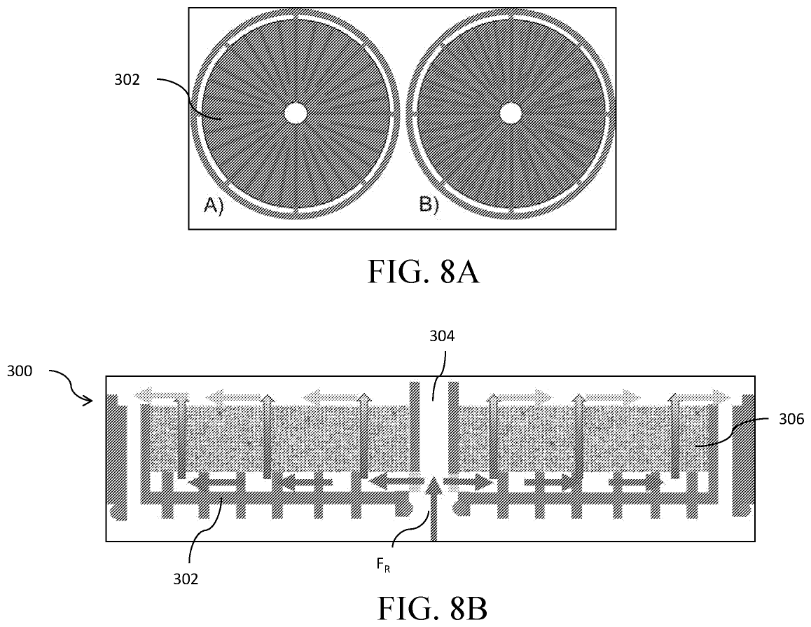

To control the delivery of media up through the fixed bed cell culture substrate, a manifold 302 is molded in which the flow velocity can vary within the manifolding (where changes in velocity are of no importance) in order to deliver even flow throughout the volume of the fixed bed(s), thus creating a uniform flow environment throughout the cell culture zones. FIG. 8A shows a plan view of the radial flow manifold 302 having support structures through which the media is manifolded and supplied to the fixed beds. On the right side of FIG. 8A, flow rates of media through the manifold 302 is depicted as varying as it spreads within the manifolding.

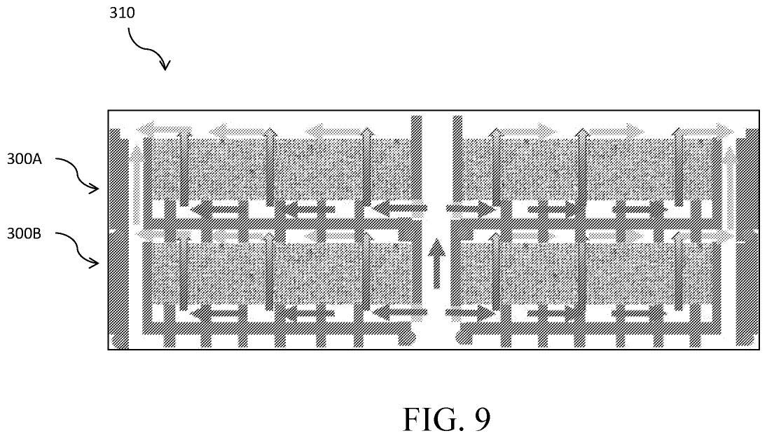

FIG. 8B shows a cross-section side view of a cell culture subunit 300 according to this embodiment. The subunit has a central flow column 304 through which media is delivered to the subunit 300. As shown by arrows FR, media flows up the central flow column 304 and then radially through the manifold 302 beneath a packed-bed substrate 306. Flow through the packed-bed substrate 306 itself is uniform due to uniform resistance within the packed bed 306 and within the manifold 302. As shown in FIG. 9, two or more of the modular subunits 300A, 300B can be stacked to form a stacked cell culture system 310. The manifold 302 thus allows for snapping together of multiple packed beds while maintaining uniform flow to all fixed bed zones.

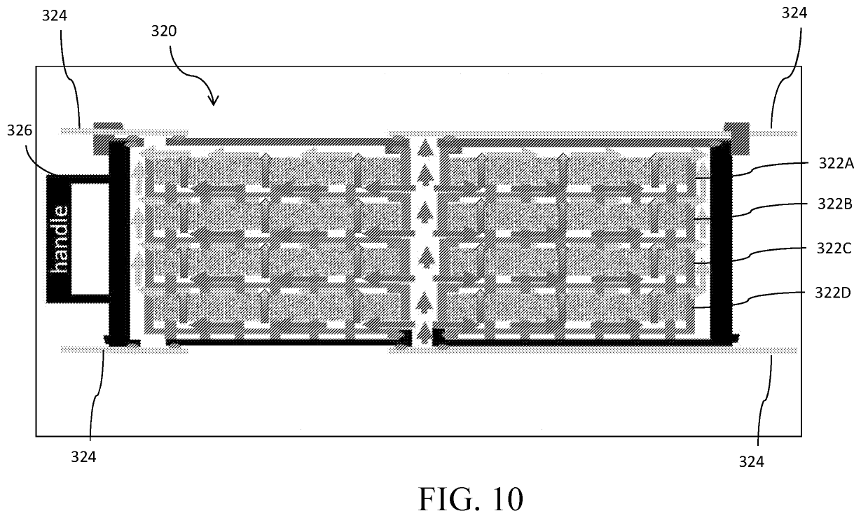

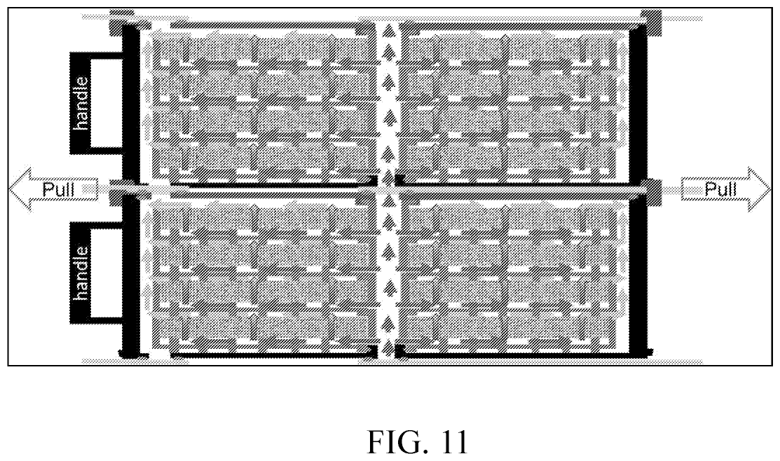

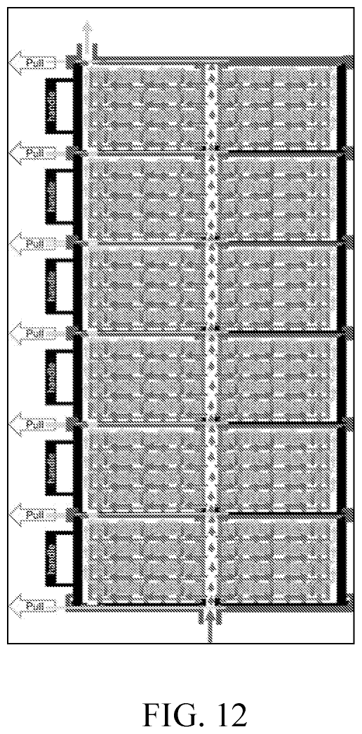

FIG. 10 shows a larger stacked cell culture system 320 consisting of four subunits 322A-322D, showing how the modularity of this design lends to direct scalability of the system. FIG. 10 also shows aseptic pull tabs 324 which can be left in place to maintain a closed system, or can be removed to further expand the modular system. The entire modular system can optionally be provided in a carrier 326 having a handle for easy handling by users. As shown in FIG. 11, two or more of these carriers 326 can be stacked and the aseptic tabs can be pulled to join the adjacent carriers 326. This arrangement allows for the system to be further expanded while maintaining modularity for easy handling. For example, FIG. 12 shows six carriers connected, with each carrier containing four cell culture subunits. The bottom and top plates of the resulting stacked system have a media inlet and a media outlet, respectively, so that media can be fed through the entire system. The resulting design is very compact, with upwards of 90% of the volume being occupied by cell culture substrate.

As an aspect of embodiments of this disclosure, a suitable cell culture substrate will enhance the flow uniformity of media through the packed bed for uniform cell seeding, uniform feeding nutrients to cells, and uniform release of cells for harvesting. Examples of such substrate materials are disclosed in U.S. Provisional Patent Application Publication Nos. 62/801,325 and 62/910,696 and PCT Application Publication No. WO 2019/104069, the contents of which are incorporated herein by reference in their entirety.

The cell culture substrate is porous to allow perfusion of cells, media, nutrients, and cell by-products through the substrate and to allow spent media with cell secreted material (e.g., recombinant protein, antibody, virus particles, DNA, RNA, sugars, lipids, biodiesel, inorganic particles, butanol, metabolic byproducts) to pass through the substrate and be harvested. Further details of the cell culture substrate according to embodiments are provided below.

The vessel or subunits disclosed herein can be plastic, glass, ceramic or stainless steel. According to some embodiments, all or part of the vessel may be made of a transparent material or may include one or more transparent windows in the outer wall of the vessel to allow for inspection of the interior of the vessel via the human eye or any of a number of sensors, probes, cameras, or monitoring units. For example, according to an aspect of some embodiments, an optical camera or Raman spectroscopy probe can be used to monitor the cell culture progress within the cavity of the vessel.

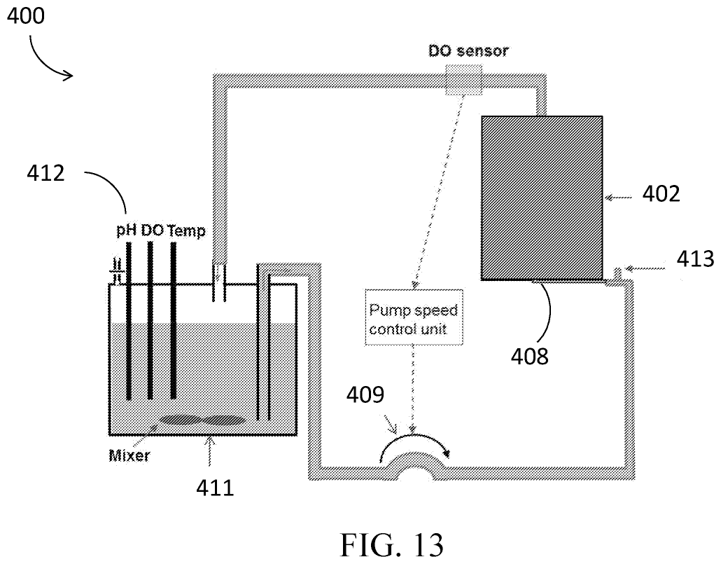

FIG. 13 shows a bioreactor 402 as described herein incorporated into a bioprocessing system 400, according to one or more embodiments. The system 400 includes a media conditioning vessel 411 for proper maintenance of cell culture media parameters such as pH, temperature, and oxygenation level, for example. Automatically controlled pump 409 is used to perfuse media through the bioreactor 402. Bioreactor inlet 413 is equipped with additional 3-way port to facilitate cell inoculation or collection of harvested cells. The system 400 may include in-line sensors, as well as the sensors in the media conditioning vessel 411.

As described above, the bioreactor according to embodiments of this disclosure can include one or more ports and sensors for monitoring and adjusting of medium and cell culture environment within the vessel. However, according to some embodiments, cell culture media sensing and conditioning can be performed in a second vessel that is external to the bioreactor. For example, FIG. 13 demonstrates the schematics of bioreactor vessel 402 that is connected to main external components comprised of a media conditioning vessel, a pump allowing the flow of media into the bioreactor and external dissolved oxygen sensor that support required process conditions for successful bioprocess. Cell culture media is conditioned in media conditioning vessel 411, where proper pH, temperature and dissolved oxygen levels are maintained. Subsequently media is perfused through the bioreactor by pump 409. Flow rate of pump 409 is integrated into a feedback loop which is automatically adjusts to maintain minimal predefined level of dissolved oxygen in media exiting the bioreactor. All transfection reagents, nutrients and additional media supplements required by given bioprocess can be introduced into bulk media and spent media can removed via the media conditioning vessel 411. At the end of the process, media can be drained from bioreactor and refilled with cells harvesting solution. After incubating the packed bed in harvesting solution for predefined time that is sufficient for cells to detach from the substrate cells are harvested by reverse flow through applying air pressure at bioreactor outlet to achieve flow rate in a range of 70 ml/cm² (cross sectional packed bed area)/min. Cells are harvested at the bioreactor 3-way port 413. Cells can also be lysed directly in the bioreactor and lysate solution containing AVV particles can be collected through 3-way port 413.

The media conditioning vessel 411 can include sensors and control components found in typical bioreactor used in the bioprocessing industry for a suspension batch, fed-batch or perfusion culture. These include but are not limited to DO oxygen sensors, pH sensors, oxygenator/gas sparging unit, temperature probes, and nutrient addition and base addition ports. A gas mixture supplied to sparging unit can be controlled by a gas flow controller for N₂, O₂, and CO₂ gasses. The media conditioning vessel 411 also contains an impeller for media mixing. All media parameters measured by sensors listed above can be controlled by a media conditioning control unit in communication with the media conditioning vessel 411, and capable of measuring and/or adjusting the conditions of the cell culture media 406 to the desired levels.

The media from the media conditioning vessel 411 is delivered to the bioreactor 402 via an inlet, which may also include an injection port for cell inoculum to seed and begin culturing of cells. The bioreactor vessel 402 may also include on or more outlets through which the cell culture media exits the vessel 402. In addition, cells or cell products may be output through the outlet. To analyze the contents of the outflow from the bioreactor 402, one or more sensors 412 may be provided in the line. In some embodiments, the system 400 includes a flow control unit for controlling the flow into the bioreactor 402. For example, the flow control unit may receive a signal from the one or more sensors 412 and, based on the signal, adjust the flow into the bioreactor 402 by sending a signal to a pump (e.g., peristaltic pump) upstream of the inlet 408 to the bioreactor 402. Thus, based on one or a combination of factors measured by the sensors 412, the pump can control the flow into the bioreactor 402 to obtain the desired cell culturing conditions.

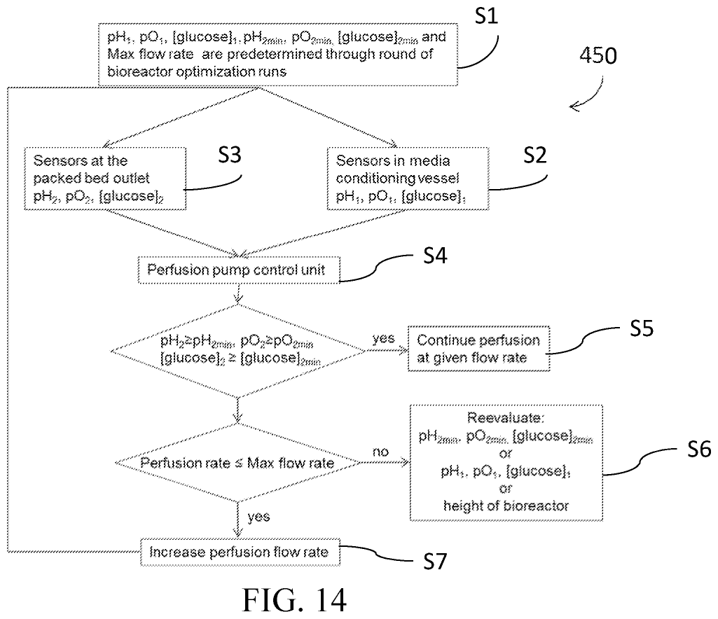

The media perfusion rate is controlled by the signal processing unit that collects and compares sensors signals from media conditioning vessel 411 and sensors located at the packed bed bioreactor outlet. Because of the pack flow nature of media perfusion through the packed bed bioreactor 402, nutrients, pH and oxygen gradients are developed along the packed bed. The perfusion flow rate of the bioreactor can be automatically controlled by the flow control unit operably connected to the peristaltic pump, according to the flow chart in FIG. 14.

FIG. 14 shows an example of a method 450 for controlling the flow of a perfusion bioreactor system, such as the system 400 of FIG. 13. According to the method 450, certain parameters of the system 400 are predetermined at step S1 through bioreactor optimization runs. From these optimization runs, the values of pH₁, pO₁, [glucose]₁, pH₂, pO₂, [glucose]₂, and maximum flow rate can be determined. The values for pH₁, pO₁, and [glucose]₁ are measured within the cell culture chamber of the bioreactor 402 at step S2, and pH₂, pO₂, and [glucose]₂ are measured by sensors 412 in the media conditioning vessel 411 at step S3 (or in the bioreactor according to embodiment discussed herein). Based on these values at S2 and S3, a perfusion pump control unit makes determinations at S4 to maintain or adjust the perfusion flow rate. For example, a perfusion flow rate of the cell culture media to the cell culture chamber may be continued at a present rate if at least one of pH₂≥pH2 min, pO₂≥pO₂ₘᵢₙ, and [glucose]₂≥[glucose]₂ min (S5). If the current flow rate is less than or equal to a predetermined max flow rate of the cell culture system, the perfusion flow rate is increased (S7). Further, if the current flow rate is not less than or equal to the predetermined max flow rate of the cell culture system, a controller of the cell culture system can reevaluate at least one of: (1) pH2 min, pO₂ₘᵢₙ, and [glucose]₂ min; (2) pH₁, pO₁, and [glucose]₁; and (3) a height of the bioreactor vessel (S6).

Embodiments of the present disclosure include bioreactors and cell culture substrates used therein, including substrates that are cell growth matrices and/or packed-bed systems for anchorage dependent cells that enable easy and effective scale-up to any practical production scale for cells or cell derived products (e.g., proteins, antibodies, viral particles). In one embodiment, a matrix is provided with a structurally defined surface area for adherent cells to attach and proliferate that has good mechanical strength and forms a highly uniform multiplicity of interconnected fluidic networks when assembled in a packed bed or other bioreactor. In particular embodiments, mechanically stable, non-degradable woven meshes can be used to support adherent cell production. Uniform cell seeding of such a matrix is achievable, as well as efficient harvesting of cells or other products of the bioreactor. In addition, the embodiments of this disclosure support cell culturing to achieve confluent monolayer or multilayer of adherent cells on disclosed matrix, and can avoid formation of 3D cellular aggregates with limited nutrient diffusion and increased metabolite concentrations. The structurally defined matrix of one or more embodiments enables complete cell recovery and consistent cell harvesting from the packed bed of bioreactor. In another embodiment of the present disclosure, a method of cell culturing is provided using bioreactors with the matrix for bioprocessing production of therapeutic proteins, antibodies, viral vaccines, or viral vectors.

In one or more embodiments, the cell culture matrix supports attachment and proliferation of anchorage dependent cells in a high volumetric density format. The matrix can be assembled and used in a bioreactor system, such as a perfused back bed bioreactor as described herein, and provide uniform cell distribution during the inoculation step, while preventing formation of large and/or uncontrollable cell aggregates inside the matrix or packed bed. Thus, the matrix eliminates diffusional limitations during operation of the bioreactor. In addition, the matrix enables easy and efficient cell harvest from the bioreactor.

The matrix can be formed with a substrate material that of a thin, sheet-like construction having first and second sides separated by a relatively small thickness. In other words, the thickness of the sheet-like substrate is small relative to the width and/or length of the first and second sides of the substrate. In addition, a plurality of holes or openings are formed through the thickness of the substrate. The substrate material between the openings is of a size and geometry that allows cells to adhere to the surface of the substrate material as if it were a two-dimensional (2D) surface, while also allowing adequate fluid flow around the substrate material and through the openings. In some embodiments, the substrate is a polymer-based material, and can be formed as a molded polymer sheet; a polymer sheet with openings punched through the thickness; a number of filaments that are fused into a mesh-like layer; or a plurality of filaments that are woven into a mesh layer. The physical structure of the matrix has a high surface-to-volume ratio for culturing anchorage dependent cells. According to various embodiments, the matrix can be arranged or packed in a bioreactor in certain ways to obtain uniform cell seeding, uniform media perfusion, and efficient cell harvest.

The cell culture substrate can be a woven mesh layer made of a first plurality of fibers running in a first direction and a second plurality of fibers running in a second direction. The woven fibers of the substrate form a plurality of openings. The size and shape of the openings can vary based on the type of weave (e.g., number, shape and size of filaments; angle between intersecting filaments, etc.). An opening can be defined by a certain width or diameter. A woven mesh may be considered, on a macro-scale, a two-dimensional sheet or layer. However, a close inspection of a woven mesh reveals a three-dimensional structure due to the rising and falling of intersecting fibers of the mesh. Thus, a thickness of the woven mesh may be thicker than the thickness of a single fiber.

The woven mesh can be comprised of monofilament or multifilament polymer fibers. In one or more embodiments, a monofilament fiber may have a diameter in a range of about 50 μm to about 1000 μm. On a microscale level, due to the scale of the fiber compared to the cells (e.g., the fiber diameters being larger than the cells), the surface of monofilament fiber is presented as regular 2D surface for adherent cells to attach and proliferate. Such fibers are woven into a mesh that has a defined pattern and a certain amount of structural rigidity. Fibers can be woven into a mesh with openings ranging from about 100 μm×100 μm to about 1000 μm×1000 μm. These ranges of the filament diameters and opening diameters are examples of some embodiments, but are not intended to limit the possible feature sizes of the mesh according to all embodiments.

The substrate mesh can be fabricated from monofilament or multifilament fibers of polymeric materials compatible in cell culture applications, including, for example, polystyrene, polyethylene terephthalate, polycarbonate, polyvinylpyrrolidone, polybutadiene, polyvinylchloride, polyethylene oxide, polypyrroles, and polypropylene oxide. Mesh substrates may have a different structure patterns or weaves, including, for example knitted, warp-knitted, or woven (plain weave, twilled weave, dutch weave, five needle weave).

The surface chemistry of the mesh filaments may need to be modified to provide desired cell adhesion properties. Such modifications can be made through the chemical treatment of the polymer material of mesh or grafting cell adhesion molecules to the filament surface. Alternatively, meshes can be coated with thin layer of biocompatible hydrogels that demonstrate cell adherence properties, including, for example, collagen or Matrigel®. Alternatively, surfaces of filament fibers of the mesh can be rendered with cell adhesive properties through the treatment processes with various types of plasmas, process gases, and/or chemicals known in the industry.

The woven mesh substrate may be provided in a number of discs with a center hole configured to surround the center column of the bioreactor described herein. A plurality of such discs can be stacked in the outer region of the bioreactor to form the packed bed.

According to some embodiments, the cell culture substrate is a dissolvable foam scaffold comprising an ionotropically crosslinked polygalacturonic acid compound selected from at least one of: pectic acid; partially esterified pectic acid, partially amidated pectic acid and salts thereof; and at least one first water-soluble polymer having surface activity.

Embodiments of this disclosure can achieve viral vector platforms of a practical size that can produce viral genomes on the scale of about 10¹⁵ to about 10¹⁸ or more viral genomes per batch. For example, in some embodiments, the viral genome yield can be about 10¹⁵ to about 10¹⁶ viral genomes or batch, or about 10¹⁶ to about 10¹⁹ viral genomes per batch, or about 10¹⁶-10¹⁸ viral genomes per batch, or about 10¹⁷ to about 10¹⁹ viral genomes per batch, or about 10¹⁸ to about 10¹⁹ viral genomes per batch, or about 10¹⁸ or more viral genomes per batch.

In addition, the embodiments disclosed herein enable not only cell attachment and growth to a cell culture substrate, but also the viable harvest of cultured cells. The inability to harvest viable cells is a significant drawback in current platforms, and it leads to difficulty in building and sustaining a sufficient number of cells for production capacity. According to an aspect of embodiments of this disclosure, it is possible to harvest viable cells from the cell culture substrate, including between 80% to 100% viable, or about 85% to about 99% viable, or about 90% to about 99% viable. For example, of the cells that are harvested, at least 80% are viable, at least 85% are viable, at least 90% are viable, at least 91% are viable, at least 92% are viable, at least 93% are viable, at least 94% are viable, at least 95% are viable, at least 96% are viable, at least 97% are viable, at least 98% are viable, or at least 99% are viable. Cells may be released from the cell culture substrate using, for example, trypsin, TrypLE, or Accutase.

Illustrative Implementations

The following is a description of various aspects of implementations of the disclosed subject matter. Each aspect may include one or more of the various features, characteristics, or advantages of the disclosed subject matter. The implementations are intended to illustrate a few aspects of the disclosed subject matter and should not be considered a comprehensive or exhaustive description of all possible implementations.

Aspect 1 pertains to a modular cell culture system comprising: a standalone cell culture subunit comprising: an interior cavity configured to house a cell culture substrate in a cell culture space, a fluid inlet configured to supply fluid to the cell culture space, a fluid outlet configured to remove fluid from the interior cavity, the interior cavity being configured to flow fluid in from the fluid inlet, through the cell culture space, and out through the fluid outlet, and at least one alignment feature disposed on at least one of a top and a bottom of the standalone cell culture subunit, wherein the at least one alignment feature is configured to align with at least one alignment feature of another standalone cell culture subunit, such that the standalone cell culture subunit is stackable with the another standalone cell culture subunit.

Aspect 2 pertains to the modular cell culture system of Aspect 1, further comprising a first flow distribution plate disposed between the fluid inlet and the cell culture space.

Aspect 3 pertains to the modular cell culture system of Aspect 2, wherein the first flow distribution plate is configured to distribute media from the fluid inlet evenly across a width of the cell culture space.

Aspect 4 pertains to the modular cell culture system of any one of Aspects 1-3, further comprising a second flow distribution plate disposed between the cell culture space and the fluid outlet.

Aspect 5 pertains to the modular cell culture system of Aspect 4, wherein the second flow distribution plate is configured to promote even flow of media out of the cell culture space.

Aspect 6 pertains to the modular cell culture system of any one of Aspects 2-5, wherein the first and second flow distribution plates define a top and a bottom of the cell culture space.

Aspect 7 pertains to the modular cell culture system of any one of Aspects 1-6, further comprising a cell culture substrate disposed within the cell culture space.

Aspect 8 pertains to the modular cell culture system of Aspect 7, wherein the cell culture substrate comprises a porous material.

Aspect 9 pertains to the modular cell culture system of Aspect 7 or Aspect 8, wherein the cell culture substrate comprises at least one of polystyrene, polyethylene terephthalate, polycarbonate, polyvinylpyrrolidone, polybutadiene, polyvinylchloride, polyethylene oxide, polypyrroles, and polypropylene oxide.

Aspect 10 pertains to the modular cell culture system of Aspect 8 or Aspect 9, wherein the cell culture substrate comprises at least one of a molded polymer lattice, a 3D-printed polymer lattice sheet, and a woven mesh sheet.

Aspect 11 pertains to the modular cell culture system of Aspect any one of Aspects 7-10, wherein the cell culture substrate comprises the woven mesh comprising one or more fibers.

Aspect 12 pertains to the modular cell culture system of Aspect 11, wherein the one or more fibers have a fiber diameter from about 50 μm to about 1000 μm, from about 50 μm to about 600 μm, from about 50 μm to about 400 μm, from about 100 μm to about 325 μm, or from about 150 μm to about 275 μm.

Aspect 13 pertains to the modular cell culture system of Aspect 11 or Aspect 12, wherein the woven mesh comprises a plurality of openings interstitial to the one or more fiber, the plurality of openings having a diameter of from about 100 μm to about 1000 μm, from about 200 μm to about 900 μm, or from about 225 μm to about 800 μm.

Aspect 14 pertains to the modular cell culture system of Aspect 7 or Aspect 8, wherein the cell culture substrate is a dissolvable foam scaffold.

Aspect 15 pertains to the modular cell culture system of Aspect 14, wherein the dissolvable foam scaffold comprises: an ionotropically crosslinked polygalacturonic acid compound selected from at least one of: pectic acid; partially esterified pectic acid, partially amidated pectic acid and salts thereof; and at least one first water-soluble polymer having surface activity.

Aspect 16 pertains to the modular cell culture system of Aspect 14 or Aspect 15, wherein the dissolvable foam scaffold comprises an adhesion polymer coating.

Aspect 17 pertains to the modular cell culture system of Aspect 16, wherein the adhesion polymer coating comprises peptides.

Aspect 18 pertains to the modular cell culture system of Aspect 17, wherein the adhesion polymer coating comprises peptides selected from the group consisting of BSP, vitronectin, fibronectin, laminin, Type I collagen, Type IV collagen, denatured collagen and mixtures thereof.

Aspect 19 pertains to the modular cell culture system of Aspect 17, wherein the adhesion polymer coating comprises Synthemax® II-SC.

Aspect 20 pertains to the modular cell culture system of any one of Aspects 1-19, further comprising a plurality of the standalone cell culture subunit, the plurality of the standalone cell culture subunits being stacked.

Aspect 21 pertains to the modular cell culture system of Aspect 20, wherein the alignment feature is an attachment feature that connects adjacent standalone cell culture subunits of the plurality of standalone cell culture subunits.

Aspect 22 pertains to the modular cell culture system of Aspect 21, wherein the attachment feature allows the adjacent standalone cell culture subunits to be releasably attached.

Aspect 23 pertains to the modular cell culture system of Aspect 21 or Aspect 22, wherein the attachment feature is at least one of an interlocking profile of the alignment features of the adjacent standalone cell culture subunits, mating components of a snap fit closure, or a threaded connection.

Aspect 24 pertains to the modular cell culture system of Aspect 4, further comprising a retaining feature disposed on an inside wall of the interior cavity, the retaining feature being configured to hold the second flow distribution plate in place against the cell culture substrate.

It will be appreciated that the various disclosed embodiments may involve particular features, elements or steps that are described in connection with that particular embodiment. It will also be appreciated that a particular feature, element or step, although described in relation to one particular embodiment, may be interchanged or combined with alternate embodiments in various non-illustrated combinations or permutations.

It is also to be understood that, as used herein the terms “the,” “a,” or “an,” mean “at least one,” and should not be limited to “only one” unless explicitly indicated to the contrary. Thus, for example, reference to “an opening” includes examples having two or more such “openings” unless the context clearly indicates otherwise.

Ranges can be expressed herein as from “about” one particular value, and/or to “about” another particular value. When such a range is expressed, examples include from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint.

All numerical values expressed herein are to be interpreted as including “about,” whether or not so stated, unless expressly indicated otherwise. It is further understood, however, that each numerical value recited is precisely contemplated as well, regardless of whether it is expressed as “about” that value. Thus, “a dimension less than 10 mm” and “a dimension less than about 10 mm” both include embodiments of “a dimension less than about 10 mm” as well as “a dimension less than 10 mm.”

Unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is no way intended that any particular order be inferred.

While various features, elements or steps of particular embodiments may be disclosed using the transitional phrase “comprising,” it is to be understood that alternative embodiments, including those that may be described using the transitional phrases “consisting” or “consisting essentially of,” are implied. Thus, for example, implied alternative embodiments to a method comprising A+B+C include embodiments where a method consists of A+B+C, and embodiments where a method consists essentially of A+B+C.

Although multiple embodiments of the present disclosure have been illustrated in the accompanying Drawings and described in the foregoing Detailed Description, it should be understood that the disclosure is not limited to the disclosed embodiments, but is capable of numerous rearrangements, modifications and substitutions without departing from the disclosure as set forth and defined by the following claims.

1. A modular cell culture system comprising:
a standalone cell culture subunit comprising:
an interior cavity configured to house a cell culture substrate in a cell culture space,
a fluid inlet configured to supply fluid to the cell culture space,
a fluid outlet configured to remove fluid from the interior cavity, the interior cavity being configured to flow fluid in from the fluid inlet, through the cell culture space, and out through the fluid outlet, and
at least one alignment feature disposed on at least one of a top and a bottom of the standalone cell culture subunit,
wherein the at least one alignment feature is configured to align with at least one alignment feature of another standalone cell culture subunit, such that the standalone cell culture subunit is stackable with the another standalone cell culture subunit.

2. The modular cell culture system of claim 1, further comprising a first flow distribution plate disposed between the fluid inlet and the cell culture space.

3. The modular cell culture system of claim 2, wherein the first flow distribution plate is configured to distribute media from the fluid inlet evenly across a width of the cell culture space.

4. The modular cell culture system of claim 1, further comprising a second flow distribution plate disposed between the cell culture space and the fluid outlet.

5. The modular cell culture system of claim 4, wherein the second flow distribution plate is configured to promote even flow of media out of the cell culture space.

6. The modular cell culture system of claim 2, wherein the first and second flow distribution plates define a top and a bottom of the cell culture space.

7. The modular cell culture system of claim 1, further comprising a cell culture substrate disposed within the cell culture space.

8. The modular cell culture system of claim 7, wherein the cell culture substrate comprises a porous material.

9. The modular cell culture system of claim 7, wherein the cell culture substrate comprises at least one of polystyrene, polyethylene terephthalate, polycarbonate, polyvinylpyrrolidone, polybutadiene, polyvinylchloride, polyethylene oxide, polypyrroles, and polypropylene oxide.

10. The modular cell culture system of claim 8, wherein the cell culture substrate comprises at least one of a molded polymer lattice, a 3D-printed polymer lattice sheet, and a woven mesh sheet.

11. The modular cell culture system of claim 7, wherein the cell culture substrate comprises the woven mesh comprising one or more fibers.

12. The modular cell culture system of claim 11, wherein the one or more fibers have a fiber diameter from about 50 μm to about 1000 μm, from about 50 μm to about 600 μm, from about 50 μm to about 400 μm, from about 100 μm to about 325 μm, or from about 150 μm to about 275 μm.

13. The modular cell culture system of claim 11, wherein the woven mesh comprises a plurality of openings interstitial to the one or more fiber, the plurality of openings having a diameter of from about 100 μm to about 1000 μm, from about 200 μm to about 900 μm, or from about 225 μm to about 800 μm.

14. The modular cell culture system of claim 7, wherein the cell culture substrate is a dissolvable foam scaffold.

15. The modular cell culture system of claim 14, wherein the dissolvable foam scaffold comprises:
an ionotropically crosslinked polygalacturonic acid compound selected from at least one of: pectic acid; partially esterified pectic acid, partially amidated pectic acid and salts thereof; and
at least one first water-soluble polymer having surface activity.

16-19. (canceled)

20. The modular cell culture system of claim 1, further comprising a plurality of the standalone cell culture subunit, the plurality of the standalone cell culture subunits being stacked.

21. The modular cell culture system of claim 20, wherein the alignment feature is an attachment feature that connects adjacent standalone cell culture subunits of the plurality of standalone cell culture subunits.

22. The modular cell culture system of claim 21, wherein the attachment feature allows the adjacent standalone cell culture subunits to be releasably attached.

23. The modular cell culture system of claim 21, wherein the attachment feature is at least one of an interlocking profile of the alignment features of the adjacent standalone cell culture subunits, mating components of a snap fit closure, or a threaded connection.

24. The modular cell culture system of claim 4, further comprising a retaining feature disposed on an inside wall of the interior cavity, the retaining feature being configured to hold the second flow distribution plate in place against the cell culture substrate.
